(19) United States
(12) Patent Application Publication
(10) Pub. No.: US 2016/0135981 A1
(43) Pub. Date: May 19, 2016

(54) FOOT ORTHOTIC FABRICATION SYSTEM AND METHODS OF OPERATING

(71) Applicant: Vincent R. Rizzo, Indianapolis, IN (US)

(72) Inventor: Vincent R. Rizzo, Indianapolis, IN (US)

(21) Appl. No.: 14/942,889

(22) Filed: Nov. 16, 2015

Related U.S. Application Data
(60) Provisional application No. 62/080,258, filed on Nov. 15, 2014.

(51) Int. Cl. A61F 5/14; A61B 5/103

(52) U.S. Cl. CPC A61F 5/14; A61B 5/1036

(57) ABSTRACT

Method and system is disclosed for manufacturing an orthopedic insert. The process includes, initializing a plurality of sensors as pixel-based information, making one or more pressure measurement from each of the plurality of sensors, analyzing and conditioning the pressure measurements to derive a pressure value for each pressure sensor, converting the pressure values into a pixel intensity value, mapping the pixel intensity values to a graphic image, determining pixel intensity values between each mapped pixel intensity value along a first axis, determining pixel intensity values along a second axis, and machining an orthotic insert based upon the graphical image.

### CROSS REFERENCE TO RELATED APPLICATIONS

This application claims the benefit of U.S. Provisional Application No. 62/080,258 filed on Nov. 15, 2014 which is hereby incorporated herein by reference.

### TECHNICAL FIELD

This disclosure relates to the field of orthopedic inserts for footwear, and more particularly to methods and systems for manufacturing orthotic inserts.

### BACKGROUND

The statements in this section merely provide background information related to the present disclosure and may not constitute prior art.

Orthopedic devices for the feet and orthopedic inserts for footwear are well known, and have been used by laypersons and podiatrists for many years to improve function, stability, and balance for the musculoskeletal system. Devices of this type range from a simple arch support to a custom formed orthotic insert to hold and control the foot.

Foot misalignment can cause discomfort and injury such as plantar fasciitis, hammertoes, bunions, Achilles tendonitis, and others. Misalignment can also cause or exacerbate knee, hip or back pain. Structural mal-alignment of the foot is generally localized to the fore foot, the rear foot, or combinations of both. These structural abnormalities may be generically classified as either of the varus or valgus type. The valgus abnormality refers specifically to a foot position, or any part thereof, wherein the joint is turned outward or everted, that is away from the body midline to an abnormal degree. The varus abnormality, on the other hand, is a condition of the foot, or any part thereof, being turned inward or inverted, that is towards the body midline to an abnormal degree.

Known orthotic inserts are typically an insole that cushions and provides general support to a user's foot. Known methods for manufacturing custom orthotic inserts are shape-based, requiring a plaster cast or foam impression directly from a user's foot. Once the casting or impression is made, they are sent to a facility specializing in orthotic manufacturing. Shape-based manufacturing can be slow and expensive often requiring multiple fittings. Accordingly, a need exists for new and improved methods and systems to manufacture orthopedic inserts.

### SUMMARY

Method and system is disclosed for manufacturing an orthopedic insert. The process includes, initializing a plurality of sensors 22 as pixel-based information, making one or more pressure measurement from each of the plurality of sensors, analyzing and conditioning the pressure measurements to derive a pressure value for each pressure sensor, converting the pressure values into a pixel intensity value, mapping the pixel intensity values to a graphic image, determining pixel intensity values between each mapped pixel intensity value along a first axis, determining pixel intensity values along a second axis, selecting an orthotic insert profile and superimposing on the graphical image, in one embodiment, and machining an orthotic insert based upon the graphical image. In one embodiment, a machining device is configured for, sizing and registering the orthotic using the customers shoe insole as a template.

Certain embodiments of the invention include a method for machining pressure based orthotics, machining a single side of a foam orthotic insert to decrease manufacturing time, and sizing and registering the orthotic using the shoe insole as a template. Certain embodiments of the invention include machining based upon a pixel intensity value for each corresponding area on the foam orthotic insert.

This summary is provided merely to introduce certain concepts and not to identify key or essential features of the claimed subject matter.

### BRIEF DESCRIPTION OF THE DRAWINGS

One or more embodiments will now be described, by way of example, with reference to the accompanying drawings, in which:

FIG. 1 schematically shows an exemplary manufacturing system, in accordance with the present disclosure;

FIG. 2 schematically shows an exemplary orthopedic measuring device, in accordance with the present disclosure;

FIG. 3 shows an exemplary process 300 for manufacturing an orthopedic insert, in accordance with the present disclosure;

FIG. 4 shows an exemplary representation of a pixel-based initialization of pixel intensity values on a graphical image, in accordance with the present disclosure;

FIG. 5 shows an exemplary representation of pressure values mapped from each sensor position to a pixel intensity value on a graphical image, in accordance with the present disclosure;

FIG. 6 shows a mapping along an x-axis of determined pixel intensity values between each mapped pixel intensity value represented in FIG. 5, in accordance with the present disclosure;

FIG. 7 shows a mapping along a y-axis of determined pixel intensity values between each mapped pixel intensity value represented in FIG. 6, in accordance with the present disclosure;

FIG. 8 shows an exemplary orthotic insert profile, in accordance with the present disclosure; and

FIG. 9 shows an exemplary desktop orthotic machining device, in accordance with the present disclosure.

### DETAILED DESCRIPTION

Throughout the specification and claims, the following terms take at least the meanings explicitly associated herein, unless the context dictates otherwise. The meanings identified below do not necessarily limit the terms, but merely provide illustrative examples for the terms. The meaning of “a,” “an,” and “the” includes plural reference, and the meaning of “in” includes “in” and “on.” The phrase “in one embodiment,” as used herein does not necessarily refer to the same embodiment, although it may. Similarly, the phrase “in some embodiments,” as used herein, when used multiple times, does not necessarily refer to the same embodiments, although it may. As used herein, the term “or” is an inclusive “or” operator, and is equivalent to the term “and/or,” unless the context clearly dictates otherwise. The term “based, in part, on”, “based, at least in part, on”, or “based upon” is not exclusive and allows for being based on additional factors not described, unless the context clearly dictates otherwise. The word “exemplary” is used herein to mean “serving as an example, instance, or illustration.” Any embodiment described herein as “exemplary” is not necessarily to be construed as preferred or advantageous over other embodiments.

Various embodiments of the present invention will be described in detail with reference to the drawings, where like reference numerals represent like parts and assemblies throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

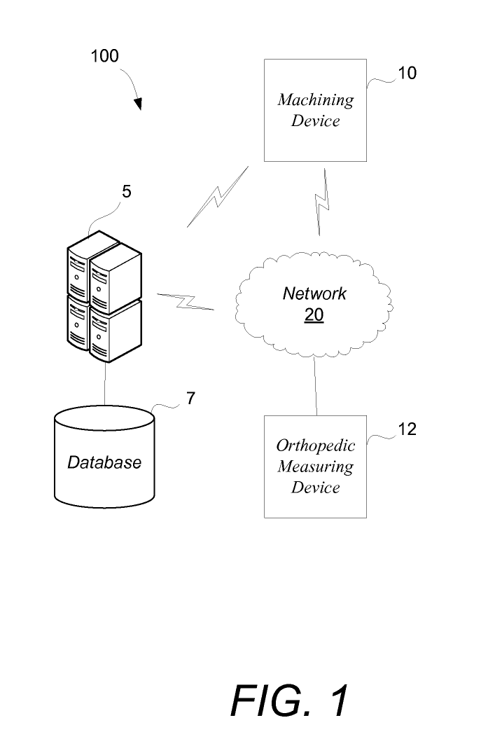

Referring now to the drawings, wherein the depictions are for the purpose of illustrating certain exemplary embodiments only and not for the purpose of limiting the same, FIG. 1 schematically shows an exemplary manufacturing system 100 that may help implement the methodologies of the present disclosure. The system 100 includes a computing device 5, a network 20, a machining device 10, and an orthopedic measuring device 12. As shown in FIG. 1, the computing device 5 may be directly communicatively connected to the machining device 10 and/or connected via the network 20. In some embodiments, the computing device 5 may be directly or indirectly connected to the orthopedic measuring device 12. In one embodiment, it is contemplated that output from the device 12 may be delivered to the computing device 5 using a removable storage drive. The device 12 may be physically connected to the network 20 or the computing device 5 during selected periods of operation without departing from the teachings herein. Components of the system 100 are shown in FIG. 1 as single elements. Such illustration is for ease of description and it should be recognized that the system 100 may include multiple additional devices 5, 10, and 12.

The network 20 may be any suitable series of points or nodes interconnected by communication paths. The network 20 may be interconnected with other networks and contain sub networks network such as, for example, a publicly accessible distributed network like the Internet or other telecommunications networks (e.g., intranets, virtual nets, overlay networks and the like). The network 20 may facilitates the exchange of data between and among the computing device 5, the machining device 10, and the orthopedic measuring device 12.

The computing device 5 may be: various embodiments of a computer or server including high-speed microcomputers, minicomputers, mainframes, and/or data storage devices. The computing device 5 preferably executes database functions including storing and maintaining a database 7 and processes requests from the device 10 to extract data from, or update, a database as described herein below. The computing device 5 may additionally provide processing functions for the machining device 10, and the orthopedic measuring device 12 as will become apparent to those skilled in the art upon a careful reading of the teachings herein.

In operation, the orthopedic measuring device 12 obtains pressure information associated with a user's foot, and provides the information to the device 5. The computing device 5 analyzes the data and generates an orthopedic insert custom to the user's foot based upon the received information. The orthopedic insert design is provided by the computer 5 to the machining device 10. The device 10 manufactures the orthopedic insert based on the design provided by the computing device 5.

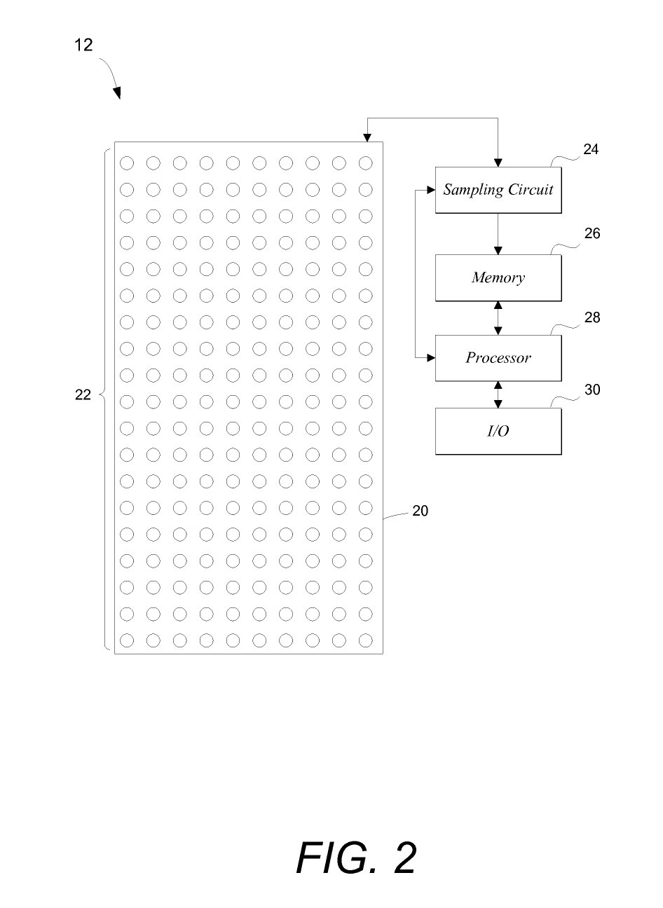

FIG. 2 schematically shows an exemplary orthopedic measuring device 12. The device 12 includes a sensor array20 that is adapted to allow a person to place the device 12 into their shoe. The device 12 includes a plurality of pressure sensors 22. The plurality of sensors 22 are disposed on the sensor array 20 in a manner sufficient to receive the entirety of a person's foot.

The sensors 22 can be piezoelectric devices, force sensitive resistors, or force capacitive transducers. The device senses pressure as a person steps or walks on the device 12 while the device is positioned in the person's shoe. The pressure sensed can be stored in a memory dedicated to each sensor or dedicated to a bank of sensors.

In one embodiment, the device 12 includes a sampling circuit 24, memory 26 and, optionally, a processor 28 and an I/O interface 30. An I/O interface 30 is preferably coupled to the processor 28. The I/O interface 30 may include one or more I/O devices such as lights, switches, a serial connection port, an infrared port, wireless capability, and/or integrated 802.11x (WiFi) wireless capability, to enable wired (e.g., USB cable) and/or wireless connection to a local or networked computer system, such as the computing device 5.

The sampling circuit 28 can control a frequency of sampling of the pressure obtained from each sensor. Each sensor 22 can provide a plurality of pressure readings for subsequent analysis. These readings are stored in the memory 26. The readings stored in the memory 26 can be transmitted to the processor or computing device 5 for further analysis. A processor 28 is optionally provided to control the sampling rate used by the sampling circuit 24, conduct data quality and integrity checks on the sensors 22 and the sensor readings, and/or analyze sensor readings.

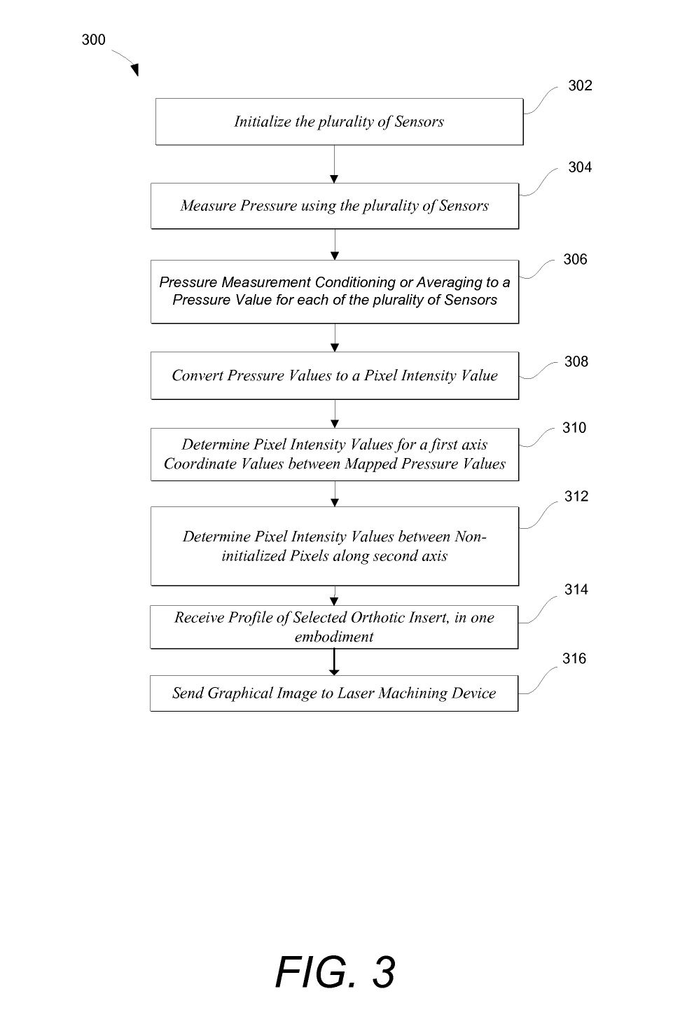

FIG. 3 shows an exemplary process 300 for manufacturing an orthopedic insert. As FIG. 3 shows, in one embodiment, the process begins at step 302 by initializing the plurality of sensors 22. The initialization process can include representing data from the sensors as pixel-based information. In one embodiment, the pixels representing each sensor are each initially set to a predetermined bit sequence representative of a pixel color or grayscale color, e.g., 0, 255, 0000000, or 1111111. Note that each pixel may be represented as a decimal-based or binary-based value indicative of the pixel intensity value.

FIG. 4 shows an exemplary representation of a pixel-based initialization of pixel intensity values on a graphical image. As shown in FIG. 4, each sensor is represented by a pixel having a preset initial intensity value. The pixels are arranged in rows and columns running either the length or width of the bitmap image. In one embodiment, the image is made up of 49 columns along the width and 125 rows along the length of the bitmap image. In one embodiment, an initial intensity value of each sensor is set at zero or 00000000 in binary. In one embodiment, an initial intensity value of each sensor is set at 1. FIG. 4 illustrates an initial intensity pixel setting for each of the sensors of 1, or 11111111 in binary although it is contemplated by the disclosure herein that the initial value may be any initial value.

Referring back to FIG. 3, subsequent to initializing each sensor, at step 304 one or more pressure measurements may be made by each of the plurality of sensors 22. In one embodiment, a user can be prompted to step on the orthopedic measuring device 12 before making the pressure measurements. In one embodiment, the sensors 22 scan 5 times per second for a duration of 30 seconds resulting in 150 measurements while the user is walking.

At step 306, the system, selects, conditions, and/or averages the pressure measurements to derive a pressure value for each pressure sensor. Pressure measurements may vary considerably while the user is walking or standing onto the sensor array 20. Hence, in one embodiment, pressure readings are used to determine whether the user's foot is stable or unmoving on the platform 20 of the orthopedic measuring device 12. In this way, a more accurate or consistent readings of pressure may be obtained. To determine whether the user's foot is stable or unmoving, multiple pressure readings may be taken over a period of time, e.g., 5 seconds. Pressure readings may be discounted while readings vary a predetermined amount between readings. Until readings are stable, e.g., fluctuations within a predetermined range between readings, readings may be discounted.

The pressure measurements may be conditioned by averaging or selecting certain readings. In one embodiment, the pressure value is an average of multiple sensor measurements made over a predetermined time period, e.g., 2 seconds. In one embodiment, the pressure value is a highest pressure reading obtained over a predetermined time period. In one embodiment, the pressure readings are selected a predetermine time period after a first reading is obtain, e.g., 2 seconds are a first recorded pressure measurement. In one embodiment, a predetermined number of the highest readings are averaged to obtain a pressure value for a sensor.

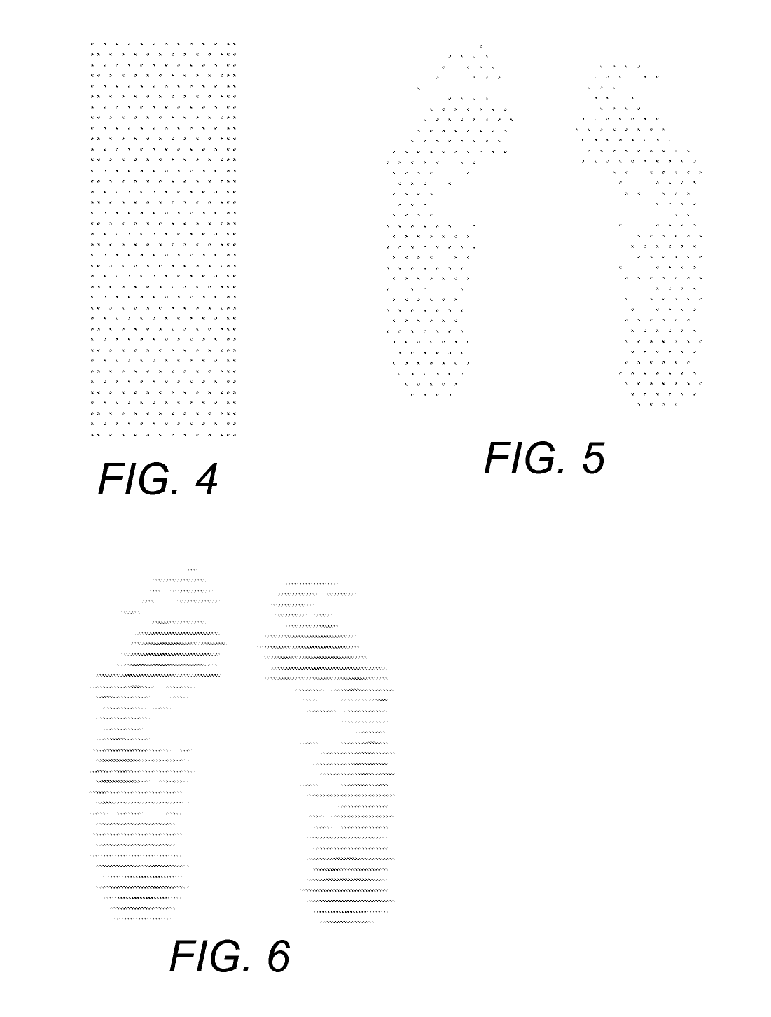

At step 308, the pressure values obtained from each sensor are each converted into a pixel intensity value and mapped to a graphic image such as a bitmap. In one embodiment, the pixel intensity values are determined based upon a predefined linear relationship between pixel color and pressure values. For example, a ‘0’ intensity value corresponds to a ‘0’ pressure reading and a ‘1’ pixel intensity value for a predefined upper bound pressure value. The coordinates on the graphic image corresponding to each sensor are predefined, in one embodiment. FIG. 5 shows an exemplary representation of pressure values mapped to each sensor via a pixel intensity value on a graphical image. In scans having pressure values vary among the sensors, the grayscale representation of each pixel on the map will vary accordingly.

In one embodiment, x-axis and y-axis coordinate values are determined for mapping a particular pressure value associated with a sensor reading. In one embodiment, a graphic image has a predefined number of cells. Each cell defines an area of pixels. For example, each cell may be a 10-pixel by 10-pixel area. Some cells are associated with a pressure sensor; each pressure sensor is associated with a unique cell. By separating the sensor values, the map represents the space between pressure readings as pixel space. In one embodiment, the pressure sensing cells are mapped using the following x-axis and y-axis formulae:

### Left Foot:

=((xOffset−i Div+i Mod)*xMap)+(xMap*2);

Y-Axis=(yOffset −(i Div+i Mod))*yMap

### Right Foot

X-Axis=(xOffset+(i Div−i Mod))*xMap;

Y-Axis=(yOffset−(i Div+i Mod)*yMap

wherein:
cNum=cell to be mapped
matrixSize=numColumns=numRows
xCells=number of sensor cells that traverses the bitmap width (determined by the physical distance between cells)
yCells=number of sensor cells that traverse the bitmap height (determined by the physical distance between cells)
xMap=width of bitmap in pixels/number of xCells
yMap=height of bitmap in pixels/number of yCells
xOffset=starting pixel for the width
yOffset=starting pixel for the height
i Div=cNum/matrixSize
i Mod=cNum modulus matrixSize

For example, in a 490 pixel by 1250 pixel bitmap, with cNum=155, xOffset=12, yOffset=44, cNum=155, i Div=155/24=6, i Mod=155 Mod 24=11, xMap=18.75, and yMap=32.04 the pressure sensing cells are mapped using the following x-axis and y-axis formulae:

### Left Foot:

x-Axis=(12−6+11)*18.75)+(18.75*2)=356

Y-Axis=(44−(6+11)*32.04=865

### Right Foot

X-Axis=12+(6−11)*18.75=131

Y-Axis=(44−(6+11)*32.04=865

In one embodiment, a plurality of pressure sensor value ranges are defined that are each associated with a pixel intensity value. In operation, a particular pressure sensor measurement or conditioned value is compared with the ranges to determine association, e.g., the measurement or value is within a particular range. The cell associated with the measurement or value is then assigned the pixel intensity value associated with the identified range that corresponds to the measurement or value. For example, after calculating the x-axis and y-axis coordinate values, the grayscale value is derived based on the average pressure value. In one embodiment, the following is used to determine the grayscale value: if reading is 0 and less than 9, the grayscale value=0; if reading is 10 and less than 17, the grayscale value=35; if reading is 18 and less than 26, the grayscale value=69; if reading is 27 and less than 50, the grayscale value=103; if reading is 51 and less than 65, the grayscale value=171; if reading is 66 and less than 82, the grayscale value=205; and if reading is 82 less than 100, the grayscale value=255. It is contemplated by the disclosure herein, that the range values and number of ranges may be increased or decreased and/or modified and may be adapted for different foam material and foam compression specifications including, e.g., foam density.

After calculating the x-axis and y-axis coordinate value and the grayscale value for each pressure cell, the pixel is mapped to the bitmap using the appropriate grayscale color values. As described herein above, FIG. 5 shows an exemplary representation of pressure values mapped to each sensor.

At step 310, a pixel intensity value of the pixels between each mapped pixel intensity value associated with pressure values for the pressure sensors is determined along a first axis. In one embodiment, the values between the each mapped pixel intensity value associated with the pressure sensors is determined based upon pixel intensity of adjacent pixels. Pixels still having the initialized value are compared to pixels in proximity that do not have the initial value. In one embodiment, initialized pixels between non-initialized pixels are counted and the difference between pixel intensity of the adjoining, non-initialized pixels is determined. The difference in pixel intensity is divided between the counted number of initialized pixels. The resulting value can be incremented or decremented for each pixel when compared with the adjacent pixel. As an example, FIG. 6 shows a mapping along an x-axis of determined pixel intensity values between each mapped pixel intensity value represented in FIG. 5.

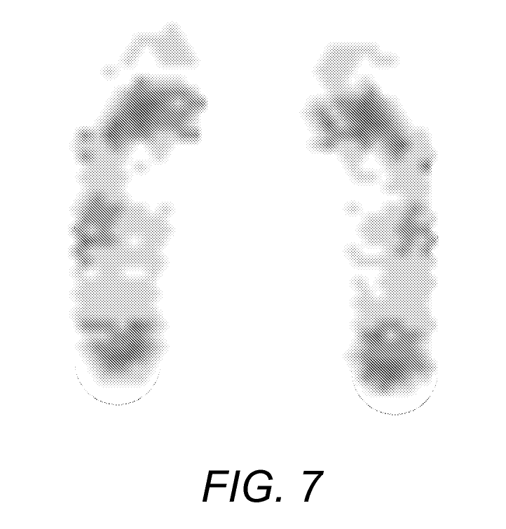

At step 312, pixel intensity values are mapped on the graphical image in the second axis between mapped pressure values represented as pixel intensity values. Similarly to step 310, pixel intensity values for initialized pixels may be determined based upon proximately located pixel intensity values. In one embodiment, the values between the each mapped pixel intensity value associated with the pressure sensors is determined based upon pixel intensity of adjacent pixels. Pixels still having the initialized value are compared to pixels in proximity that do not have the initial value. In one embodiment, initialized pixels between non-initialized pixels are counted and the difference between pixel intensity of the adjoining, non-initialized pixels is determined. The difference in pixel intensity is divided between the counted number of initialized pixels. The resulting value can be incremented or decremented for each pixel when compared with the adjacent pixel. As an example, FIG. 7 shows a mapping along a y-axis of determined pixel intensity values between each mapped pixel intensity value represented in FIG. 6.

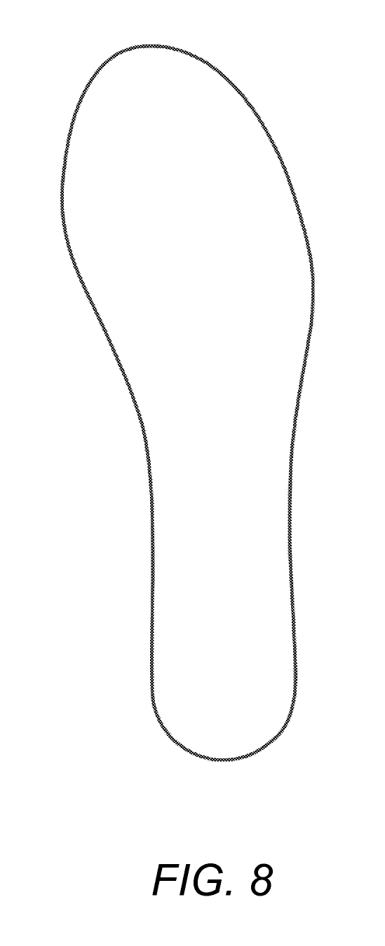

In one embodiment, at step 314, an orthotic insert profile is selected and added to the graphical image. FIG. 8 shows an exemplary orthotic insert profile. The orthotic insert profile may be selected based upon a shape and size of the non-initialized pixels. In one embodiment, the system may select an orthotic insert profile based upon a shoe size and shape selection or determination. The orthotic insert profile may include a lateral and heel alignment lines. In one embodiment, a predetermined number of orthotic insert profiles are stored and selectable. The orthotic insert profiles may be categorized by sex or gender, e.g., men's and women's profiles, and may be additionally be associated with shoe sizes.

Additional information stored within the orthopedic measuring device 12 including sensor memory 26 may be transmitted to the computing device 5 at various times. In one embodiment, additional data including one or more of sensor serial numbers, firmware version, collection site ID, consumer ID, and shoe size is transmitted to the computing device 5. In various embodiments using a milling machine, the sensor size information is sent to the mill where the mill machines a heel and lateral features that may be used to align and register the shoe insole from the persons shoe to be used as a template to insure proper fit of the orthotic, helping achieve desirous alignment to the shoe and registration of the features that were machined onto the orthotic with the person's foot and shoe.

In one embodiment, a static array may be used to store the heel alignment feature, in rows, for the sensor size. For example: HeelArray [ ]={1120, 1140, 1160, 1180, 1200}.

In one embodiment, a y-axis value for the lateral alignment feature may be stored in a static parameter used to determine the lateral feature for both the right and left foot. In various embodiments using a profile, the stored orthotic insert profiles may be associated with shoe sizes and styles which may be indexed to a predetermined Heel Array. Lateral and heel alignment lines are plotted to the bitmaps using values stored in the arrays and values computed programmatically based upon measured sensor readings. The lateral and heel lines are used to properly align a stored orthotic insert profile. Each stored orthotic insert profile may be associated with a shoe outline that may be used to identify an appropriate orthotic insert profile. As an example, the orthotic insert profile shown in FIG. 8 would be superimposed over the mapped pixel intensity values shown in exemplary FIG. 7 to generate a completed graphical image for machining.

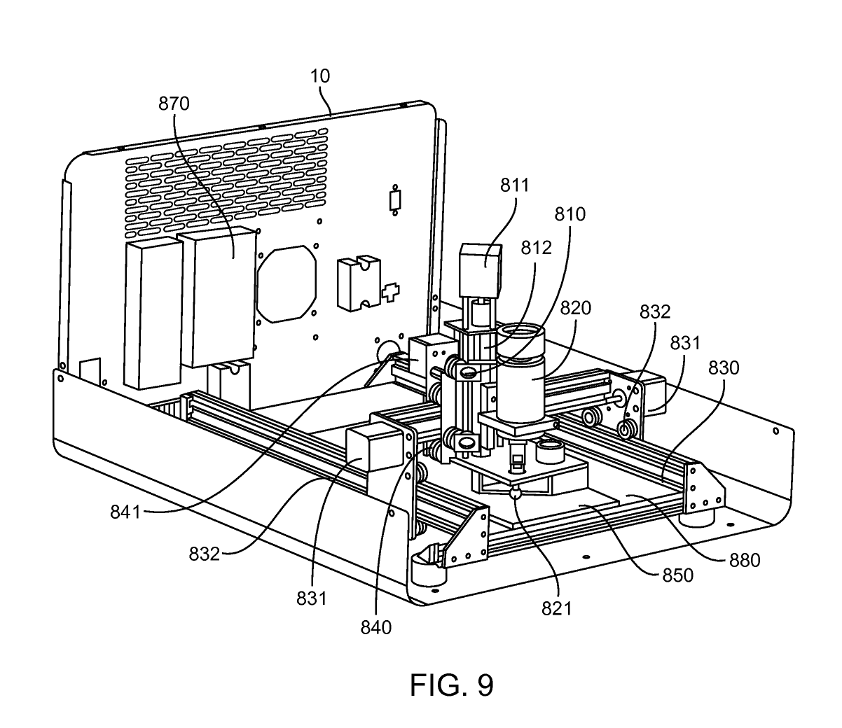

FIG. 9 shows an exemplary representation of a machining device 10 capable of manufacturing pressure based orthotics from grayscale values within a bitmap image. A piece of foam material 850 is positioned on a platform 880 which is machined to produce the pressure based orthotic. The machining device 10 includes a spindle 820 with an attached foam cutting tool. 821. The spindle 820 and cutting tool 821 are attached to the X axis 830, Y axis 840, and the Z axis 810 rails that position the spindle at a precise location on the foam 850. The machining device 10 is capable of machining smooth transitions of varying heights, based on the grayscale value within the bitmap image at specific locations that correlate to the rows and columns also within the bitmap image. The rows, columns, and height information is transmitted to the machining device 10 via the computing device 5. The smooth transitions of varying heights between columns are achieved by adjusting the Z axis 810, based on the grayscale values, while simultaneously moving the Yaxis 840 from right to left based on column locations.

After all the columns have been processed, the spindle is moved to the next row, based on the bitmap image, where the columns are processed as above. The spindle 820 is positioned via a series of motors and linear movement components attached to each axis rail. The motors may be servo or stepper driven. The linear movement components may be a linear screw or belt and pulley. In one embodiment, the X and Y axis 830 & 840 use stepper motors 831/841 with belt and pulleys and the Z axis 810 uses a stepper motor 811 with linear screw 812. In one embodiment, the smooth transitions are machined into the foam based on a series of 49 grayscale values measured at each column within the bitmap image and transmitted to the machining device 10 from the computing device 5.

The microprocessor 870 in the machining device 10 sequentially processes each grayscale value by determining the difference in grayscale values between the columns, computing an incremental height for each Y axis 840 step, and moving the Z axis 810 in unison with the Y axis 840 movement. The following algorithm is exemplary: (1) Computing device 5 sends an initial command to machining device to start machining; (2) While not all 125 rows have been processed do the following: (a) Machining device sends a command to computing device to send a row of grayscale values; (b) Computing device sends a row of grayscale values; (c) Machining device sets the last gray scale value to zero; (d) Machining device sets the column number=zero; (3) While not all 49 columns have been processed do the following: (i) Machining device stores the current gray scale value; (ii) If the current gray scale value is the same as the last value don't move the Z axis; (iii) If the current gray scale value is less than the last value, move the Z axis up; (iv) GrayScaleOffest=last value−current value; (v) If the current gray scale value is greater than the last value, move the Z axis down; (vi) GrayScaleOffset=current value−last value; (vii) Machining device stores the current value in the last value; (viii) Z axis increment=grayscaleOffset/colIndex[colNum]; (ix) For each Y step, the machining device moves the Z axis (either up or down, i.e., depth) based on the Z axis increment value; (x) Machining device increments the spindle to the next column; (4) After processing all columns, the machining device sends a command to computing device to send next row; (5) After all rows and columns are processed, the machining device cuts the heel and lateral features for alignment. (6) Process is complete.

At step 316, the orthotic foam material is placed in the machine for orthotic machining. In one embodiment, if the machining device 10 is a laser, the completed graphical image is sent to the laser for machining. If the machining device 10 is a 3 axis orthotic router mill, the grayscale values are sent over to the mill in a series of 49 byte values. In one embodiment, the bitmap image is traversed in the X axis every tenth pixel constituting a row. Then the grayscale value for every tenth pixel in the Y axis, constituting a column, is computed and sent to the mill. Pixel intensity values of the graphical image are translated to machining. For example, pixel intensity values associated with high pressure are translated by the machining device as an area requiring the removal of more foam. In one embodiment, machining is executed on a single, top side of the orthotic foam material, reducing the necessary machining to produce an effective orthotic. The disclosure has described certain preferred embodiments and modifications thereto. Further modifications and alterations may occur to others upon reading and understanding the specifications. Therefore, it is intended that the disclosure not be limited to the particular embodiment(s) disclosed for carrying out this disclosure.

1. A method of manufacturing an orthotic shoe insert, comprising:
receiving foot pressure information from a plurality of pressure sensors;
generating a graphical-based representation based upon the received foot pressure information;
determining machining coordinates and machining depth based upon the graphical-based representation; and
machining the orthotic shoe insert based upon the determining.

2. The method of claim 1, where the machining is executed on only a single side of the orthotic shoe insert.

3. The method of claim 1, wherein the generating the graphical-based representation based upon the received foot pressure information comprises converting the received foot pressure information into pixel-based information.

4. The method of claim 3, wherein the pixel-based information includes color information and location information associated with the graphical-based representation.

5. The method of claim 4, wherein the graphical-based representation includes pixel cells comprising an area of pixels, wherein each pressure sensor of the plurality of pressure sensors has a unique pixel cell on the graphical-based representation.

6. The method of claim 5, further comprising:
determining color information for pixels not associated with a location on the graphical-based representation that is associated with one of the plurality of pressure sensors based upon: (1) proximity to the location on the graphical-based representation that is associated with one of the plurality of pressure sensors and (2) the color that is associated with the unique location of the one of the plurality of pressure sensors.

7. The method of claim 6, wherein the proximity is determined based upon a single, predefined axis.

8. The method of claim 5, further comprising:
iteratively, identifying a first pixel not associated with a location on the graphical-based representation that is associated with one of the plurality of pressure sensors along a first axis;
identifying a second pixel associated with one of the plurality of pressure sensors along the first axis on a first side of the first pixel;
identifying a third pixel associated with one of the plurality of pressure sensors along the first axis on a second side of the first pixel; and
determining color information for the first pixel based upon proximity to the second pixel, proximity to the third pixel, color information associated with the second pixel, and color information associated with the third pixel.

9. The method of claim 8, further comprising:
iteratively, identifying a pixel not having color information;
identifying a closest upper pixel having color information and a closest lower pixel having color information, each upper and lower pixel along a second axis; and
determining color information for the identified pixel not having color information based upon proximity to the upper pixel, proximity to the lower pixel, color information associated with the upper pixel, and color information associated with the lower pixel.

10. The method of claim 1, wherein the determining machining coordinates based upon the graphical-based representation comprises . . .

11. A method of manufacturing an orthotic shoe insert, comprising:
receiving foot pressure information from a plurality of pressure sensors;
generating a graphical-based representation based upon the received foot pressure information, wherein the graphical-based representation comprises a plurality of pixels;
determining machining coordinates based upon the graphical-based representation; and
machining the orthotic shoe insert based upon the determining.

12. The method of claim 11, wherein the plurality of pixels are arranged in rows and columns, and wherein the graphical-based representation forms a predefined shape.

13. The method of claim 11, wherein the foot pressure information comprises information from each of the plurality of pressure sensors.

14. The method of claim 13, wherein the information from each of the plurality of pressure sensors are taken when a user is walking.

15. The method of claim 13, wherein the information from each of the plurality of pressure sensors is an average of a plurality of measurements from each of the plurality of pressure sensors.

16. The method of claim 13, further comprising:
conditioning the information from each of the plurality of pressure sensors based upon predefined sensor value boundaries and whether measurements from the plurality of pressure sensors were obtained when a user was walking.

17. The method of claim 13, wherein the information from each of the plurality of pressure sensors is based upon a maximum reading from each of the plurality of pressure sensors.

18. The method of claim 11, wherein the generating a graphical-based representation based upon the received foot pressure information comprises:
mapping the graphical-based representation into a plurality of cells having identifiable coordinates, each cell comprising a plurality of pixels, wherein each cell corresponds one of the plurality of pressure sensors;
identifying a first pressure sensor of the plurality of pressure sensors and a first associated cell;
converting the received foot pressure information for the first pressure sensor into a converted pixel intensity value; and
changing an initial pixel intensity value of the first associated cell to the converted pixel intensity value.

19. The method of claim 18, wherein the generating a graphical-based representation based upon the received foot pressure information further comprises:
identifying a first unassociated cell unassociated with any of the plurality of pressure sensors;
identifying at least one cell associated with a pressure sensor proximate to the identified first unassociated cell; and
determining a pixel intensity value for the first unassociated cell based upon pixel intensity values of the identified least one cell associated with a pressure sensor proximate to the identified first unassociated cell.

20. The method of claim 19, wherein the identified least one cell associated with a pressure sensor proximate to the identified first unassociated cell is located along a first axis, and wherein the generating a graphical-based representation based upon the received foot pressure information further comprises:
determining pixel intensity values for unassociated cells along the first axis at associated with rows or columns of cells associated with one or more of the plurality of pressure sensors; and
determining pixel intensity values for remaining unassociated cells based upon the determined pixel intensity values for the unassociated cells along the first axis at associated with rows or columns of cells associated with one or more of the plurality of pressure sensors proximate to a selected cell of the remaining unassociated cells along a column or row.

21. The method of claim 18, wherein the converting the received foot pressure information for the first pressure sensor into a converted pixel intensity value comprises:
defining a plurality of pressure sensor value ranges, each pressure sensor value range associated with a pixel intensity value;
determining which range of pressure sensor value ranges that the identified first pressure sensor is within; and
assigning a pixel intensity value to the first associated cell based upon the determining which range of pressure sensor value ranges that the identified first pressure sensor is within.

22. A method of manufacturing an orthotic shoe insert, comprising:
receiving foot pressure information from a plurality of pressure sensors;
generating a graphical-based representation based upon the received foot pressure information, wherein the graphical-based representation comprises a plurality of pixels grouped into a plurality of cells;
identifying associated cells corresponding to the received foot pressure information from the plurality of pressure sensors;
converting the received foot pressure information from the plurality of pressure sensors into pixel intensity values for the identified cells within the graphical-based representation;
identifying unassociated cells unassociated with the received foot pressure information from the plurality of pressure sensors;
determining pixel intensity values of the unassociated cells based upon the pixel intensity values of the associated cells;
determining machining coordinates based upon the graphical-based representation; and
machining the orthotic shoe insert based upon the determining.

23. The method of claim 22, wherein the predefined proximate range is a cell number between identified associated cells, wherein the pixel intensity values of the unassociated cells are a determined by:
calculating a difference between pixel intensities of a first associated cell and a second associated cell, wherein the first and second associated cells are within a row or column of the graphical-based representation;
counting a number of unassociated cells between the first and second associated cells; and
determining pixel intensity values for the unassociated cells between the first and second associated cells based upon the number of the unassociated cells between the first and second associated cells and the difference between pixel intensities of a first associated cell and a second associated cell.

24. The method of claim 23, further comprising:
identifying remaining unassociated cells having an initial pixel intensity value; and
further determining pixel intensity values for the remaining unassociated cells based upon pixel intensity values of adjacent cells.
